(19) United States
(12) Patent Application Publication
(10) Pub. No.: US 2013/0230579 A1
(43) Pub. Date: Sep. 5, 2013

(54) IMMUNOGENIC COMPOSITION COMPRISING NEISSERIA MENIGITIDIS MACROPHAGE INFECTIVITY POTENTIATOR PROTEIN

(71) Applicants: John Edward Heckels, Southampton (GB); Myron Christodoulides, Southampton (GB)

(72) Inventors: John Edward Heckels, Southampton (GB); Myron Christodoulides, Southampton (GB)

(73) Assignee: UNIVERSITY OF SOUTHAMPTON, Southampton (GB)

(21) Appl. No.: 13/821,069

(22) PCT Filed: Sep. 9, 2011
(86) PCT No.: PCT/GB11/51691

§ 371 Date: May 15, 2013

(30) Foreign Application Priority Data
Sep. 9, 2010 (GB) 1014967.2

(51) Int. Cl. A61K 39/095

(52) U.S. Cl. CPC A61K 39/095
USPC 424/450; 530/350; 424/250.1

(57) ABSTRACT

An immunogenic composition comprising the Nm-MIP protein or an immunogenic part thereof or an analogue thereof, wherein the composition is capable of eliciting an immune response when administered to a human or non-human animal, and uses thereof. A pharmaceutical composition or a vaccine composition comprising the Nm-MIP protein or an immunogenic part thereof or an analogue thereof.

The present invention relates to immunogenic compositions for use in eliciting an immune response to a pathogenic organism, and in particular, to immunogenic compositions capable of eliciting protective immune responses.

Infections caused by Neisseria meningitidis (meningococcus) are significant causes of mortality and morbidity worldwide. Despite the success of the meningococcal serogroup C conjugate vaccines introduced into the routine immunisation schedules of developed countries (Borrow and Miller 2006 Expert Review of Vaccines 5: 851-857), no such vaccines exist for serogroup B infection. The conjugate strategy that has been successful for both serogroup C and serogroup A capsule is unlikely to be effective for serogroup B due to structural similarities between the B capsule and the human foetal brain NCAM, thus raising the possibility of inducing auto-immune responses. As a result, serogroup B vaccine development has focused on the use of isolated sub-capsular outer membrane (OM) proteins for epidemic control, but these are complex and protection is PorA sero-subtype specific (Hoist et al 2009 Vaccine 27: B3-B12). Thus, the goal is to identify antigens present in the OM that are conserved and capable of inducing cross-protective antibody responses. Recently, reverse vaccinology has led to the development of a pentavalent recombinant protein vaccine, 5CMBV, which has been used in clinical trials with success when administered with OM (Giuliani et al 2006 Proc. Nat. Acad. Sci. 103: 10834-10839). In addition, many proteins present in the OM, such as the major porins PorA and PorB, the opacity protein Opc, fHBP and others have also been prepared as recombinant proteins and investigated as experimental vaccines. However, many of these antigens are variable and unsuitable as vaccine candidates.

An aim of the present invention is provide one or more compositions which can be used to elicit a protective immune response against Neisseria meningitidis (N. meningitidis).

The present invention relates to novel compositions, and in particular, to novel immunogenic compositions, comprising the N. meningitidis-macrophage infectivity potentiator (Nm-MIP) protein, and to the use of these compositions to elicit an immune response against N. meningitidis.

According to a first aspect, the present invention provides an immunogenic composition comprising the Nm-MIP protein or an immunogenic part thereof or an analogue thereof, wherein the composition is capable of eliciting an immune response when administered to a human or non-human animal.

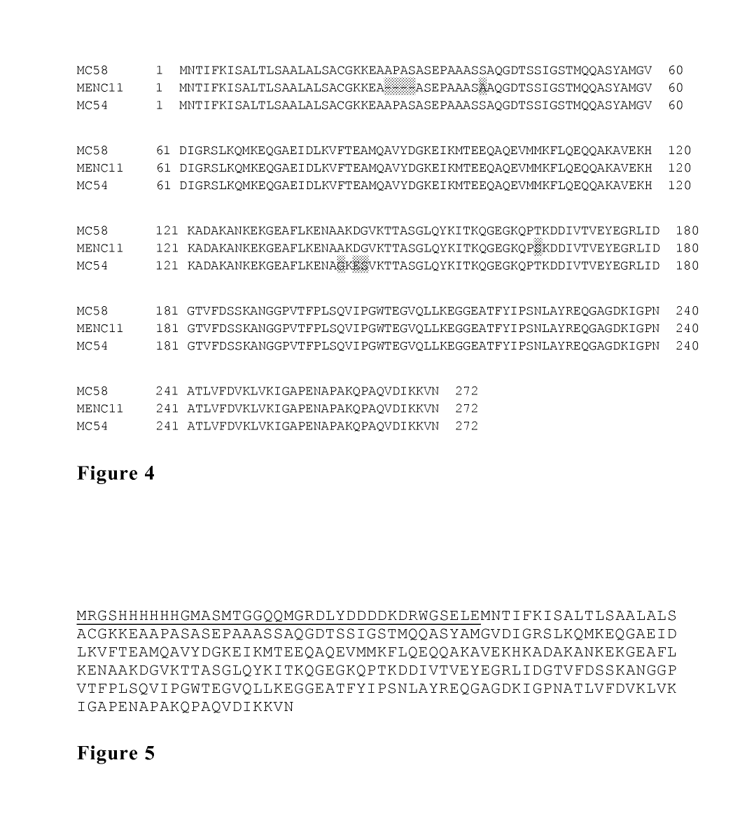

The Nm-MIP protein is present in the outer membrane of N. meningitidis. The Nm-MIP protein is encoded by the Nm-MIP gene the sequence of which can be accessed from the NCBI website (http://www.ncbi.nlm.nih.gov/) using accession number NMB1567. With reference to the Neisseria strain MC58 the Nm-MIP protein is a 272 amino acid protein as shown in FIG. 4 (Seq ID No: 1). The Nm-MIP protein may be a protein of any of Seq ID No: 1, Seq ID No: 2, Seq ID No: 3, Seq ID No: 4, Seq ID No: 5, Seq ID No: 6, Seq ID No: 7, Seq ID No: 8, Seq ID No: 9, Seq ID No: 10, Seq ID No: 11 or Seq ID No: 12, or a protein with 50%, 60%, 70%, 80%, 85%, 90%, 95%, 97%, 97%, 98% 99% or more, preferably 80% or more, preferably 85% or more, preferably 90% or more, preferably 95% or more, sequence homology with the Nm-MIP protein of Seq ID No: 1, Seq ID No: 2, Seq ID No: 3, Seq ID No: 4, Seq ID No: 5, Seq ID No: 6, Seq ID No: 7, Seq ID No: 8, Seq ID No: 9, Seq ID No: 10, Seq ID No: 11 or Seq ID No: 12.

As used in the present application, an “analogue” can include a variant in which one or more residues are added, deleted, inserted or substituted, while having no material effect on the function of the protein. That is, an analogue in accordance with one aspect of the present invention should be capable of inducing an antibody or T-cell response to the Nm-MIP protein. A residue (or residues) may be added or deleted from either end of the protein, deleted from within the protein, inserted within the protein, or substituted for one or more of the residues within the protein. As would be understood by a person of ordinary skill in art, one or more protein residues may be added, deleted, inserted or substituted while still maintaining the function of the protein. For example, as many as five or more residues may be added to or removed from either end of a protein, or inserted into a protein, and be considered a protein analogue within the context of the present invention. In a further example, a conservative substitution of one or more residues within a protein may result in a protein analogue. As would be well understood to the skilled artisan, a conservative substitution includes a substitution of one amino acid residue with another amino acid residue having one or more similar chemical properties, such as polarity, charge, hydrophobicity, or aromaticity, for example.

The Nm-MIP protein, or an immunogenic part thereof, or an analogue thereof, may further comprise additional sequence, for example, for use in purification of the protein. For example, if the protein is a recombinant protein the protein may include a his-tag sequence for use in protein purification. The protein used in the method of the invention may have the sequence of Sequence ID No: 2 (FIG. 5).

As used in the present application, an “immunogenic part” may include any part of the Nm-MIP protein that can be used to elicit a protective immune response when administered to a human or non-human animal. The composition may also comprise an analogue of an immunogenic part of the Nm-MIP protein.

Reference herein to the Nm-MIP protein is intended to refer to the Nm-MIP protein of Seq ID No: 1. Seq ID No: 2, Seq ID No: 3, Seq ID No: 4, Seq ID No: 5, Seq ID No: 6, Seq ID No: 7, Seq ID No: 8, Seq ID No: 9, Seq ID No: 10, Seq ID No: 11 or Seq ID No: 12 or to a protein with 50%, 60%, 70%, 80%, 85%, 90%, 95%, 97%, 97%, 98% 99% or more, preferably 80% or more, preferably 85% or more, preferably 90% or more, preferably 95% or more, sequence homology with the Nm-MIP protein of Seq ID No: 1, Seq ID No: 2, Seq ID No: 3, Seq ID No: 4, Seq ID No: 5, Seq ID No: 6, Seq ID No: 7, Seq ID No: 8, Seq ID No: 9, Seq ID No: 10, Seq ID No: 11 or Seq ID No: 12.

Reference to percentage homology relates to the percent identity between two aligned sequences. The percent identity refers to the residues in two proteins which are the same, when the protein sequences are aligned for maximum correspondence and when inversions and translocations are accounted for.

Preferably the percent identity ignores any conservative differences between the aligned sequences which do not affect function. The percent identity between aligned sequences can be established by using well-established tools (such as the BLAST algorithm—Basic Local Alignment Search Tool; Altschul et al., (1990) J Mol Biol. 215:403-10

Variations in percent identity may be due, for example, to amino acid substitutions, insertions or deletions. Amino acid substitutions may be conservative in nature.

In one embodiment, the immunogenic composition of the invention comprises the Nm-MIP protein of Sequence ID No 1, or a protein with 50%, 60%, 70%, 80%, 85%, 90%, 95%, 97%, 97%, 98% 99% or more, preferably 80% or more, preferably 85% or more, preferably 90% or more, preferably 95% or more, sequence homology with the Nm-MIP protein of Sequence ID No: 1, or an analogue thereof, wherein the composition is capable of eliciting an immune response when administered to a human or non-human animal.

Alternatively, the immunogenic composition may comprise the Nm-MIP protein of Seq ID No: 2, Seq ID No: 3, Seq ID No: 4, Seq ID No: 5, Seq ID No: 6, Seq ID No: 7, Seq ID No: 8, Seq ID No: 9, Seq ID No: 10, Seq ID No: 11 or Seq ID No: 12, or a protein with 50%, 60%, 70%, 80%, 85%, 90%, 95%, 97%, 97%, 98% 99% or more, preferably 80% or more, preferably 85% or more, preferably 90% or more, preferably 95% or more, sequence homology with the Nm-MIP protein of Seq ID No: 2, Seq ID No: 3, Seq ID No: 4, Seq ID No: 5, Seq ID No: 6, Seq ID No: 7, Seq ID No: 8, Seq ID No: 9, Seq ID No: 10, Seq ID No: 11 or Seq ID No: 12, or an analogue thereof.

In another embodiment, the immunogenic composition of the invention comprises the Nm-MIP protein of Sequence ID No 1, wherein the composition is capable of eliciting an immune response when administered to a human or non-human animal.

In a further embodiment, the immunogenic composition of the invention comprises the Nm-MIP protein of Sequence ID No 2, or a protein with 50%, 60%, 70%, 80%, 85%, 90%, 95%, 97%, 97%, 98% 99% or more, preferably 80% or more, preferably 85% or more, preferably 90% or more, preferably 95% or more, sequence homology with the Nm-MIP protein of Sequence ID No: 2, or an analogue thereof, wherein the composition is capable of eliciting an immune response when administered to a human or non-human animal.

Alternatively, the immunogenic composition may comprise the Nm-MIP protein of Seq ID No: 2, Seq ID No: 3, Seq ID No: 4, Seq ID No: 5, Seq ID No: 6, Seq ID No: 7, Seq ID No: 8, Seq ID No: 9, Seq ID No: 10, Seq ID No: 11 or Seq ID No: 12, or a protein with 50%, 60%, 70%, 80%, 85%, 90%, 95%, 97%, 97%, 98% 99% or more, preferably 80% or more, preferably 85% or more, preferably 90% or more, preferably 95% or more, sequence homology with the Nm-MIP protein of Seq ID No: 2, Seq ID No: 3, Seq ID No: 4, Seq ID No: 5, Seq ID No: 6, Seq ID No: 7, Seq ID No: 8, Seq ID No: 9, Seq ID No: 10, Seq ID No: 11 or Seq ID No: 12, or an analogue thereof.

In another embodiment, the immunogenic composition of the invention comprises the Nm-MIP protein of Sequence ID No 2, wherein the composition is capable of eliciting an immune response when administered to a human or non-human animal.

The compositions of the present invention are particularly suitable for preparing immunogenic compositions, such as vaccines, for use in the prevention of N. meningitidis infection. However, it will be appreciated that the compositions may be used in the prevention of infection by other related pathogens, or pathogens with a similar MIP protein. Such pathogens include Neisseria gonorrhoeae.

Preferably the immune response elicited by a composition of the invention is effective against N. meningitidis serogroup B strains, the immune response may also be effective against N. meningitidis serogroup A strains and/or N. meningitidis serogroup C strains and/or N. meningitidis serogroup W135 strains and/or N. meningitidis serogroup Y strains. The immune response elicited may also or alternatively be effective against Neisseria gonorrhoeae (N. gonorrhoeae).

Preferably the immune response elicited by a composition of the invention is effective against one or more N. meningitidis strains, preferably against at least two, at least three, at least 4, at least 5 or more strains of N. meningitidis.

Preferably the immune response elicited by the composition of the invention affects the ability of N. meningitidis and/or N. gonorrhoeae to infect an immunised animal. Preferably the ability of N. meningitidis and/or N. gonorrhoeae to infect a human immunised with the composition of the invention is impeded or prevented. This may be achieved in a number ways. The immune response elicited may recognise and destroy N. meningitidis and/or N. gonorrhoeae. Alternatively, or additionally, the immune response elicited may impede or prevent the replication of N. meningitidis and/or N. gonorrhoeae. Alternatively, or additionally, the immune response elicited may impede or prevent N. meningitidis and/or N. gonorrhoeae causing disease in the human or non-human animal. Preferably the immune response elicited is directed to at least N. meningitidis serogroup B.

The Nm-MIP protein may be recovered from N. meningitidis and/or it may be produced recombinantly and/or it may a synthetic product, for example produced by in vitro peptide synthesis or in vitro translation.

The composition of the invention may also comprise a further one or more antigens, in addition to the Nm-MIP protein or an immunogenic part thereof or an analogue thereof. The further antigens may also be derived from N. meningitidis, and may be capable of eliciting an immune response directed to N. meningitidis.

The composition may be used to elicit/produce a protective immune response when administered to a subject. The protective immune response may cause N. meningitidis to be killed upon infecting the subject, or it may prevent or inhibit N. meningitidis from replicating and/or from causing disease.

The composition may be used as a prophylactic or a therapeutic vaccine directed to N. meningitidis, and in particular N. meningitidis serogroup B. The composition may also or alternatively be used as a prophylactic or a therapeutic vaccine directed to N. gonorrhoeae.

According to a further aspect, the invention provides a pharmaceutical composition comprising the Nm-MIP protein or an immunogenic part thereof or an analogue thereof and a pharmaceutically acceptable carrier or excipient.

Preferably the pharmaceutical composition comprises a composition according to the first aspect of the invention.

Preferably the pharmaceutical composition is capable of producing a protective immune response to N. meningitidis and/or N. gonorrhoeae.

The phrase “producing a protective immune response” as used herein means that the composition is capable of generating a protective response in a host organism, such as a human or a non-human mammal, to whom it is administered. Preferably a protective immune response protects against subsequent infection by N. meningitidis and/or N. gonorrhoeae. The protective immune response may eliminate or reduce the level of infection by reducing replication of N. meningitidis, and/or N. gonorrhoeae, or by affecting the mode of action of N. meningitidis, and/or N. gonorrhoeae, to reduce disease. Preferably the protective response is directed to at least N. meningitidis serogroup B.

Suitable acceptable excipients and carriers will be well known to those skilled in the art. These may include solid or liquid carriers. Suitable liquid carriers include water and saline. The proteins of the composition may be formulated into an emulsion or they may be formulated into biodegradable microspheres or liposomes.

Preferably a composition of the invention is incorporated into liposomes or detergent micelles for administration.

Preferably, the Nm-MIP protein or immunogenic part thereof or analogue thereof in the composition of the invention is folded into its native configuration.

The composition may further comprise an adjuvant, wherein an adjuvant enhances the protective efficacy of the composition. Suitable adjuvants will be well known to those skilled in the art, and may include emulsifiers, muramyl dipeptides, pyridine, aqueous adjuvants such as aluminum hydroxide, chitosan-based adjuvants, monophosphoryl Lipid A and any of the various saponins, oils, and other substances known in the art, such as Amphigen, LPS, bacterial cell wall extracts, bacterial DNA, CpG sequences, synthetic oligonucleotides and combinations thereof. Other suitable adjuvants can be formed with an oil component, such as a single oil, a mixture of oils, a water-in-oil emulsion, or an oil-in-water emulsion. The oil may be a mineral oil, a vegetable oil, or an animal oil. Mineral oils are liquid hydrocarbons obtained from petrolatum via a distillation technique, and are also referred to in the art as liquid paraffin, liquid petrolatum, or white mineral oil. Suitable animal oils include, for example, cod liver oil, halibut oil, menhaden oil, orange oil and shark liver oil, all of which are available commercially. Suitable vegetable oils, include, for example, canola oil, almond oil, cottonseed oil, corn oil, olive oil, peanut oil, safflower oil, sesame oil, soybean oil, and the like. Freund's Complete Adjuvant (FCA) and Freund's Incomplete Adjuvant (FIA) are two common adjuvants that are commonly used in vaccine preparations, and are also suitable for use in the present invention. Both FCA and FIA are water-in-mineral oil emulsions; however, FCA also contains a killed Mycobacterium sp.

In a preferred embodiment the adjuvant used in a composition of the invention is aluminium hydroxide and/or monophosphoryl Lipid A.

In a further preferred embodiment the adjuvant used in a composition of the invention is monophosphoryl Lipid A.

In one embodiment no adjuvant is included in a composition of the invention.

The composition may also comprise polymers or other agents to control the consistency of the composition, and/or to control the release of the protein from the composition.

The composition may also comprise other agents such as diluents, which may include water, saline, glycerol or other suitable alcohols etc; wetting or emulsifying agents; buffering agents; thickening agents for example cellulose or cellulose derivatives; preservatives; detergents; antimicrobial agents; and the like.

Preferably the active ingredients in the composition are greater than 50% pure, usually greater than 80% pure, often greater than 90% pure and more preferably greater than 95%, 98% or 99% pure. With active ingredients approaching 100% pure, for example about 99.5% pure or about 99.9% pure, being used most often.

The composition of the present invention may be used as vaccine against infections caused by N. meningitidis, in particular serogroup B and possibly also serogroup A and/or serogroup C and/or serogroup W135 and/or serogroup Y and/or N. gonorrhoeae. The composition may be used as a vaccine directed to N. meningitidis or other invasive meningococcal diseases including septicaemia or septic shock, and/or as a vaccine directed to N. gonorrhoeae. The vaccine may be administered prophylactically to those at risk of exposure to N. meningitidis and/or N. gonorrhoeae, and/or therapeutically to persons who have already been exposed to N. meningitidis and/or N. gonorrhoeae.

Preferably, if the composition is used as a vaccine, the composition comprises an immunologically effective amount of antigen wherein the composition comprises the Nm-MIP protein or an immunogenic part thereof or an analogue thereof. An “immunologically effective amount” of an antigen is an amount that when administered to an individual, either in a single dose or in a series of doses, is effective for treatment or prevention of infection by N. meningitidis and/or N. gonorrhoeae. This amount will vary depending upon the health and physical condition of the individual to be treated and on the antigen. Determination of an effective amount of an immunogenic or vaccine composition for administration to an organism is well within the capabilities of those skilled in the art.

A composition according to the invention may be for oral, systemic, parenteral, topical, mucosal, intramuscular, intravenous, intraperitoneal, intradermal, subcutaneous, intranasal, intravaginal, intrarectal, transdermal, sublingual, inhalation or aerosol administration.

The composition may be arranged to be administered as a single dose or as part of a multiple dose schedule. Multiple doses may be administered as a primary immunisation followed by one or more booster immunisations. Suitable timings between priming and boosting immunisations can be routinely determined.

A composition according to the invention may be used in isolation, or it may be combined with one or more other immunogenic or vaccine compositions, and/or with one or more other therapeutic regimes.

Compositions of the invention may be able to induce serum bactericidal antibody responses and elicit antibodies which mediate opsonophagocytosis after being administered to a subject. These responses are conveniently measured in mice and the results are a standard indicator of vaccine efficacy.

The compositions of the invention may also, or alternatively, be able to elicit an immune response which neutralises bacterial proteins or other molecules, thereby preventing them from having their normal function and preventing or reducing disease progression without necessarily destroying the pathogenic organism/bacteria, in this case to N. meningitidis and/or N. gonorrhoeae.

According to a further aspect, the present invention provides the use of the Nm-MIP protein or an immunogenic part thereof or an analogue thereof in the preparation of a medicament for eliciting an immune response. The medicament may be used for the prophylactic or therapeutic vaccination of subjects against N. meningitidis and/or N. gonorrhoeae. The medicament may be a prophylactic or a therapeutic vaccine. The vaccine may be for meningitis, septicaemia and/or septic shock caused by N. meningitidis.

According to a yet further aspect, the invention provides a composition comprising the Nm-MIP protein or an immunogenic part thereof or an analogue thereof for use in generating an immune response to N. meningitidis and/or N. gonorrhoeae. The immune response may be prophylactic or therapeutic. The composition may be for use as a vaccine.

According a still further aspect, the present invention provides a method of protecting a human or non-human animal from the effects of infection by N. meningitidis and/or N. gonorrhoeae comprising administering to the human or non-human animal a composition according to any other aspect of the invention. The composition may be a vaccine.

According to another aspect, the invention provides a method for raising an immune response in a human or non-human animal comprising administering a pharmaceutical composition according to the invention to the human or non-human animal. The immune response is preferably protective. The method may raise a booster response in a patient that has already been primed. The immune response may be prophylactic or therapeutic.

One way to check the efficacy of a therapeutic treatment comprising administration of a composition according to the invention involves monitoring for N. meningitidis and/or N. gonorrhoeae infection after administration of the composition. One way to check the efficacy of a prophylactic treatment comprising administration of a composition according to the invention involves monitoring immune responses to N. meningitidis and/or N. gonorrhoeae after administration of the composition.

According to another aspect, the invention provides the use of the Nm-MIP protein or an immunogenic part thereof or an analogue thereof in the preparation of a medicament for use in the immunisation of human or non-human mammals against infection by N. meningitidis and/or N. gonorrhoeae.

According to a further aspect the invention provides a kit for use in inducing an immune response in an organism, comprising an immunogenic or vaccine composition according to the invention and instructions relating to administration.

In addition to their use as vaccines, compositions according to the invention may be useful as diagnostic reagents and as a measure of the immune competence of a vaccine.

While all the statements of invention and preferable features discussed above refer to N. meningitidis, the skilled man will appreciate that they could equally apply to N. gonorrhoeae.

It will be appreciated that optional features applicable to one aspect of the invention can be used in any combination, and in any number. Moreover, they can also be used with any of the other aspects of the invention in any combination and in any number. This includes, but is not limited to, the dependent claims from any claim being used as dependent claims for any other claim in the claims of this application.

Preferred embodiments of the present invention will now be described, merely by way of example, with reference to the following figures and examples.

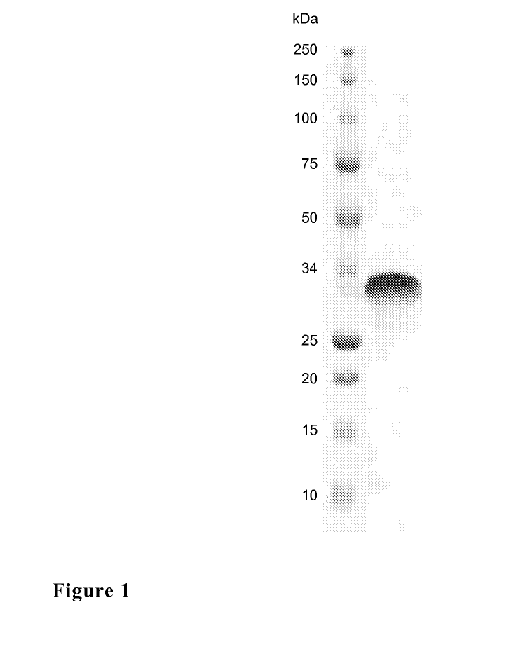

FIG. 1—is a gel showing recombinant Nm-MIP purified to homogeneity using nickel affinity chromatography under native conditions. A single band of molecular weight ˜33 kDa is shown in the second column.

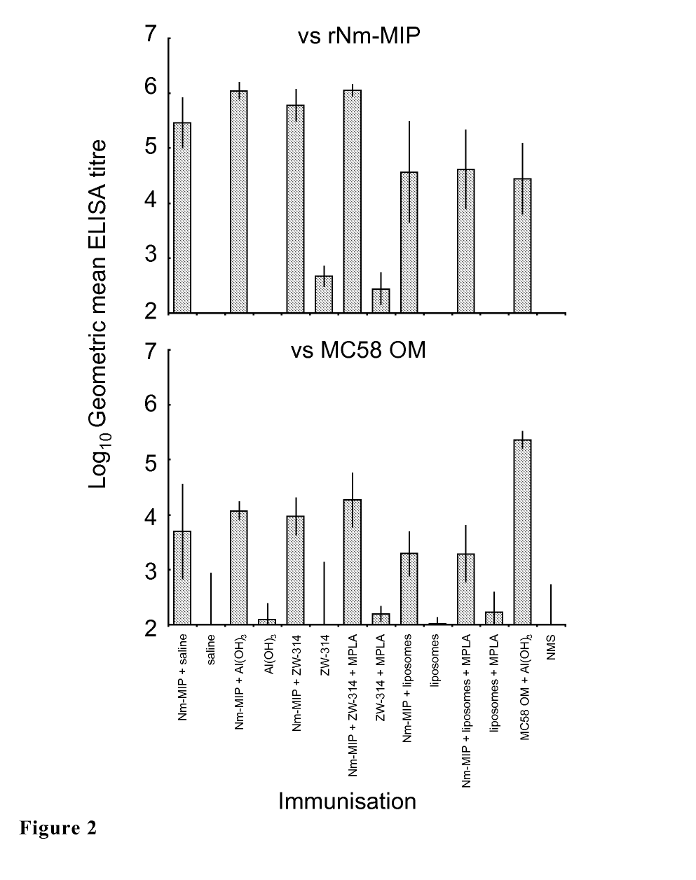

FIG. 2—shows the results of ELISA studies on the reactivity of antisera raised against rNm-MIP formulae. Antisera from individual animals, raised against rNm-MIP in various formulae, were reacted against homologous rNm-MIP protein and OM from MC58. The columns represent the geometric mean reciprocal ELISA titres (n=5 animals per group) and the error bars the 95% confidence limits.

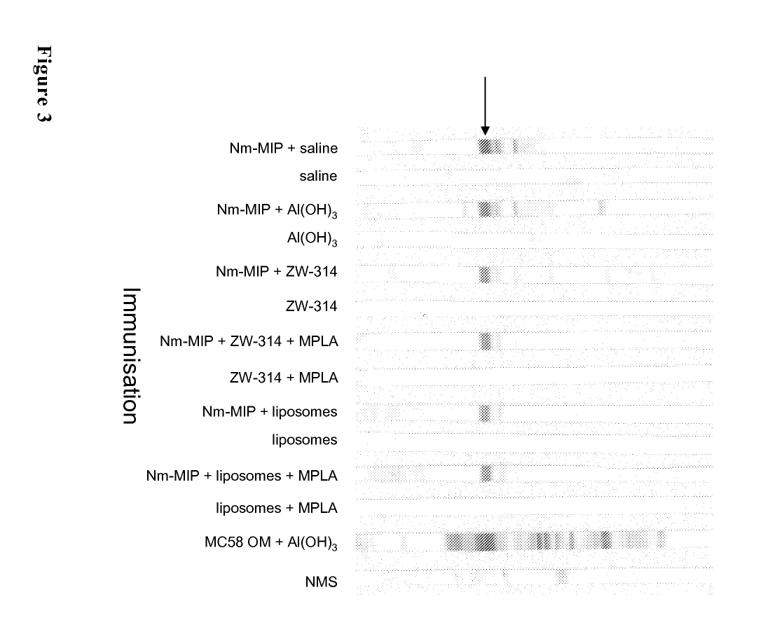

FIG. 3—shows the results of the analysis of the reactivity of antisera raised against rNm-MIP on a western blot. Whole meningococcal cell lysates were separated by SDS-PAGE, the proteins transferred to nitrocellulose and reacted with pooled antisera of each group of animals immunized with the different rNm-MIP formulae. The arrow denotes the position of the immuno-reactive Nm-MIP protein (˜29 kDa) and similar results were obtained with OM preparation.

FIG. 4—details the sequence of the Nm-MIP protein isolated from N. meningitidis strain MC58 (Sequence ID No: 1), strain MENC11 and strain MC54.

FIG. 5—details the sequence of the Nm-MIP protein isolated from N. meningitidis strain MC58 of FIG. 4 with a 39aa sequence added which include a his-tag sequence. This sequence is also referred to as Sequence ID No: 2.

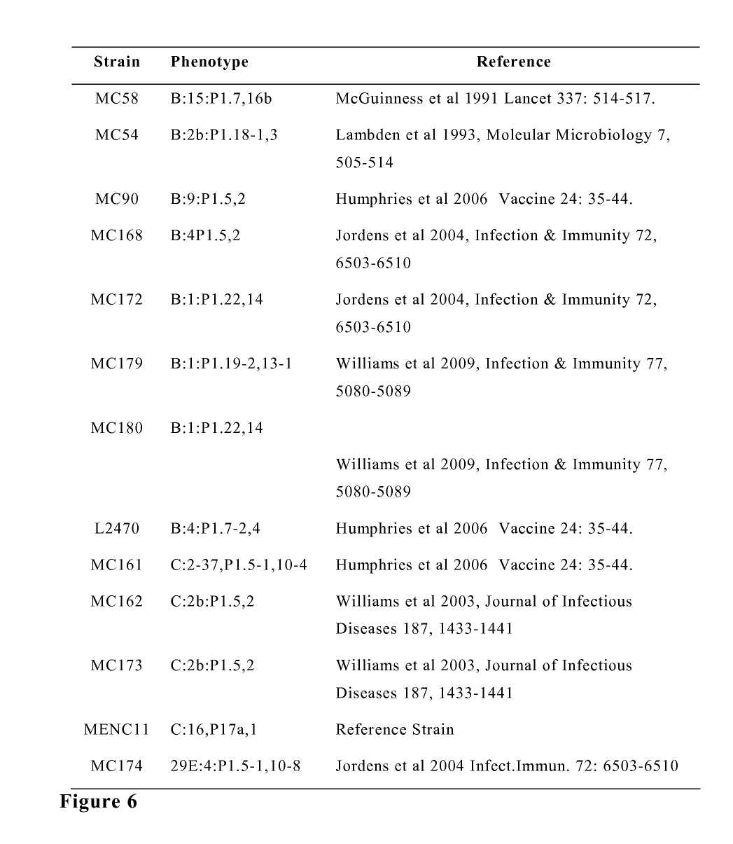

FIG. 6—lists the meningococcal strains used in the study.

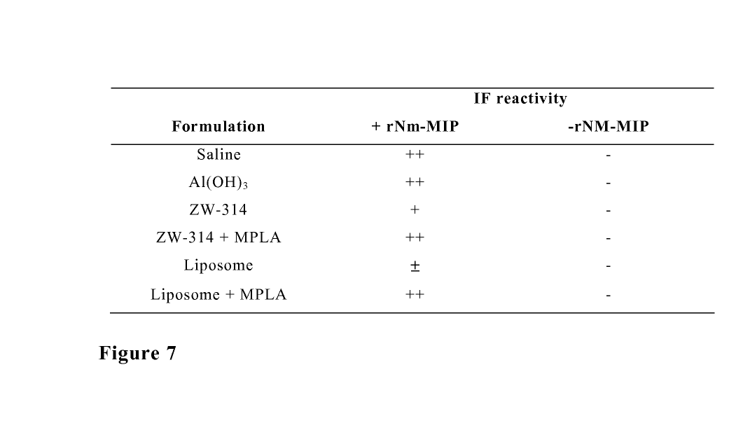

FIG. 7—details the reactivity of antisera raised against rNm-MIP on whole MC58 meningococcal cells determined by immuno-fluorescence. Pooled antisera of each group of animals immunized with the different rNm-MIP formulae was reacted (1/100 dilution) with fixed whole meningococcal cells and antibody binding detected with fluorescence conjugate. IF reactivity is indicated as follows: non-reactivity (−), weak (±), medium (+) and strong (++) determined as previously described (Jolley et al 2001. Infect. Immun. 69: 3809-3916.). For comparison, antisera raised to OM showed very strong (++) reactivity with whole cells.

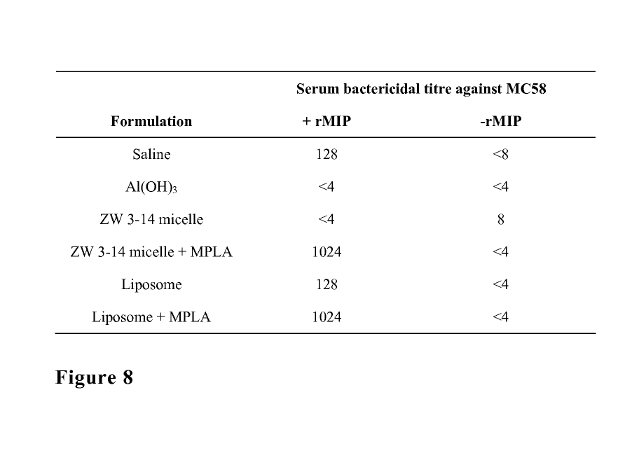

FIG. 8—details the bactericidal activity of pooled antisera raised against rNm-MIP protein. Titres are expressed as the reciprocal of the highest dilution at which 50% killing was observed. Titres for normal mouse sera and sera from mice immunised with MC580M were <8 and 20,000 respectively. Data are representative of 3 independent measurements of bactericidal activity of all pooled serum samples.

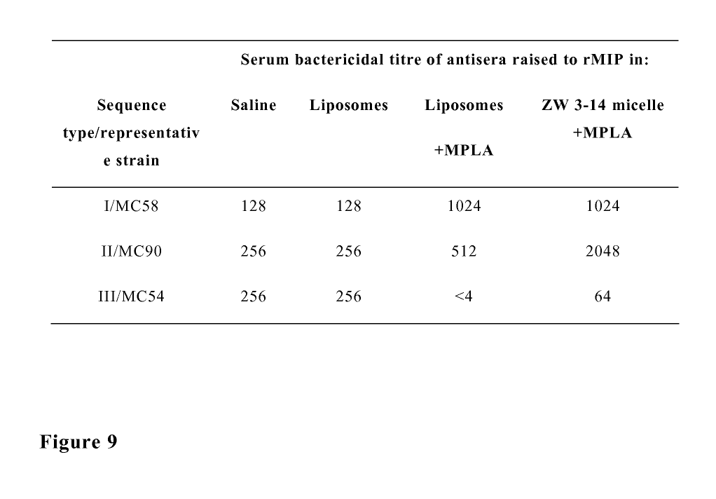

FIG. 9—details the cross-strain bactericidal activity of rNm-MIP antisera. Pooled antisera raised to rNm-MIP from MC58 were tested against heterologous strains with variations in MIP sequence. The titres are expressed as the reciprocal of the highest dilution at which 50% killing was observed. Titres for normal mouse sera and sera from mice immunised with controls without rNm-MIP were <4. Data are the median values for SBA from 3 or more independent measurements of bactericidal activity of all pooled serum samples.

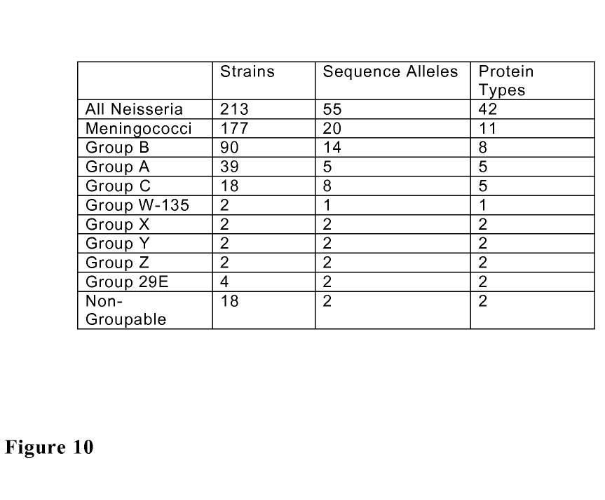

FIG. 10—details MIP gene alleles and protein diversity in the Neisseria species. Gene sequences were obtained from the NEIS1487 locus database and this study, translated and the protein sequences aligned.

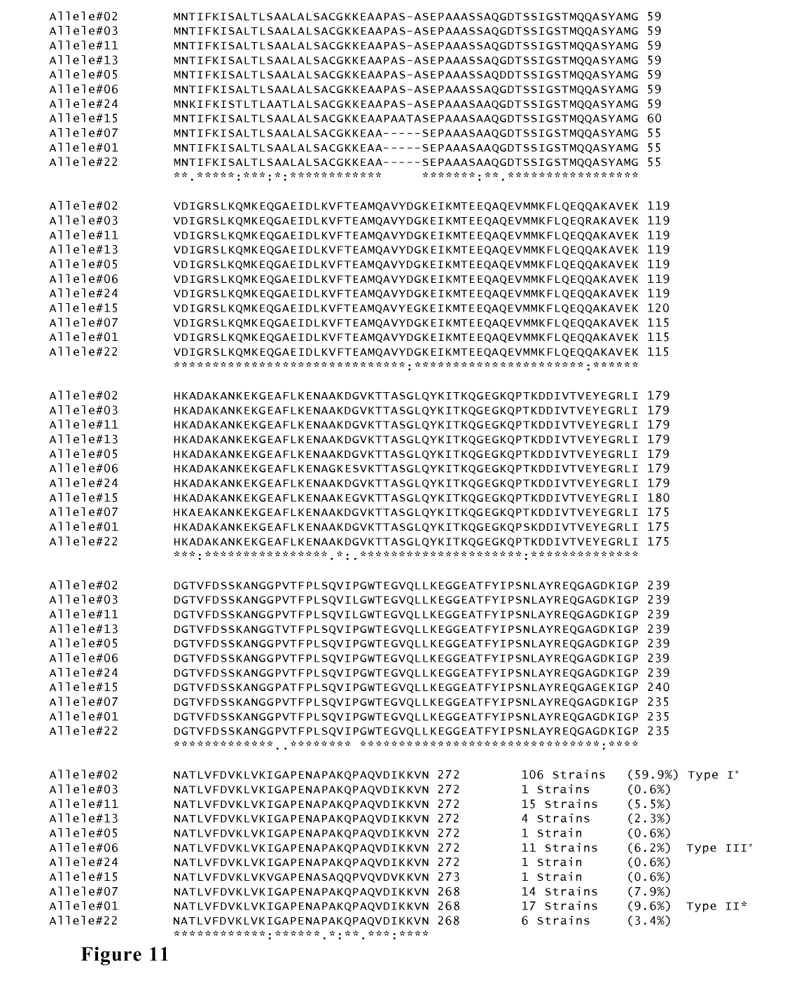

FIG. 11—shows a comparison of MIP protein types from strains of N. meningitidis. The unique translated protein sequences from 177 strains were aligned with ClustalW, where the original alleles differed only by synonymous base substitutions the sequence is denoted with the lowest allele number from that group of sequences.

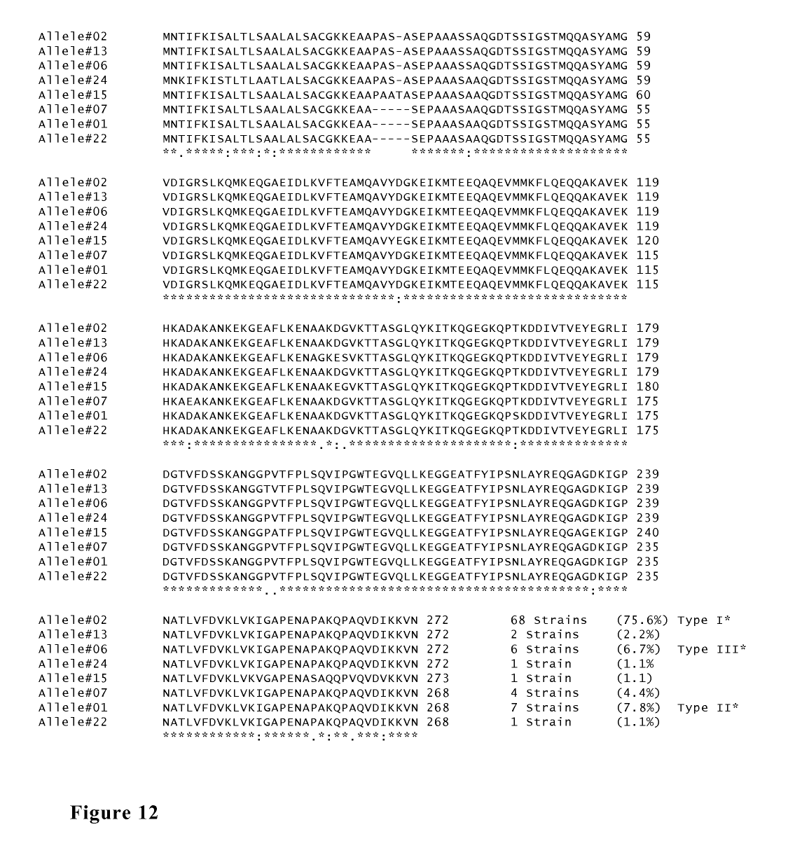

FIG. 12—shows a comparison of MIP protein types from serogroup B N. meningitidis. The unique translated protein sequences from 90 strains were aligned with ClustalW, where the original alleles differed only by synonymous base substitutions the sequence is denoted with the lowest allele number from that group of sequences.

### MATERIALS AND METHODS

### Bacterial Strains, Vectors, and Growth Conditions

N. meningitidis strain MC58 (B:15:P1.7,16b: Cap+Opa+Opc+PorA+PorB+Pil+[type IV; Class I] Rmp⁺LOS⁺) was isolated from an outbreak of meningococcal infection that occurred in Stroud, Gloucestershire in the mid-1980's (McGuinness et al 1991 Lancet 337: 514-517). Other strains included in the study are listed in FIG. 5. Meningococcal strains were grown on supplemented proteose-peptone agar (GC-agar) at 37° C. for 18 h in an atmosphere containing 5% (vol/vol) CO₂. Outer membranes of strain MC58 were prepared by extraction of whole cells by lithium acetate as previously described (Williams et al 2007 Infect. Immun. 75: 1364-1372).

The pRSETA plasmid (Invitrogen, UK), maintained in Eschericia coli DH5α (Invitrogen, UK), was used for cloning genes encoding Nm-MIP. E. coli BL21(DE3)pLysS (Invitrogen, UK) was transformed by recombinant pRSETA for protein expression. Both Luria-Bertani (LB) broth and agar were used for growing E. coli. For protein expression, transformed BL21(DE3)pLysS bacteria were cultured on SOB medium supplemented with the ampicillin (50 μg/ml; Sigma-Aldrich, Poole, UK) and chloramphenicol (30 μg/ml; Sigma-Aldrich, UK).

Cloning and Expression of the Nm-MIP Gene in E. coli

i) Genomic DNA of Strain MC58:

This was extracted by alkaline lysis as described previously (Christodoulides et al 2001. Recombinant proteins in vaccine development, p. 167-180. In A. J. Pollard and M. C. J. Maiden (ed) Meningococcal Vaccines, Humana Press, Totowa, N.J.) Briefly, a single colony of MC58 was resuspended in 10 μL ultra-high quality (UHQ) water; meningococci were lysed by the addition of 10 μl of 0.25M potassium hydroxide followed by boiling for 5 min. The sample was neutralized by the addition of 10 μl of 0.5M Tris-HCl buffer (pH 7.5) and the final volume was adjusted to 130 μl with the addition of UHQ water. Extracted genomic DNA was stored at −20° C. until used.

ii) Primer Design and Amplification of Target DNA Sequence.

The gene sequence encoding Nm-MIP (NMB1567) was accessed from the NCBI website (http://www.ncbi.nlm.nih.gov/) and the Seqbuilder (Lasergene, DNASTAR) program was used to identify restriction enzyme absent sites leading to the choice of XhoI and HindIII enzymes for cloning experiments. Two primers were designed to amplify by PCR only the mature Nm-MIP protein, with the forward primer incorporating the restriction site for XhoI (ctcgag) after the overhang sequence (NMB1567F:5′-ggctatctcgagatgaacaccattttcaaaat-3′) and the reverse primer incorporating the restriction site for HindIII (aagctt) (NMB1567R: 5′-ggctataagcttctattaatttacttttttgatgt-3′). For the forward primer, the 20 nucleotide bases following the enzyme restriction site represents the first 20 bases of the target DNA sequence and in the reverse primer the sequence represent the reverse complement of the last 20 bases. Amplification of the target DNA sequence from MC58 genomic DNA was done using the 2× Phusion™ PCR master mix (Finnzymes, UK) which contains a proof-reading enzyme (Phusion DNA polymerase). A 50 μl reaction volume contained 250 of 2× Phusion™ PCR master mix, 2.5 μl each of forward and reverse primers (10 μM; Eurogentec, UK), 19 μl of UHQ water and 1 μl of template DNA. The PCR was done in a Biometra T3 thermocycler (UK), with 30 cycles of denaturation (98° C., 10 s), annealing (60° C., 30 s) and extension (72° C., 25 s), and a final extension at 72° C., 5 min.

The PCR product was subjected to electrophoresis (1% (wt/vol) agarose gel) and the visible band excised and purified using the Wizard® SV gel and PCR clean-up system (Promega, UK) according to the manufacturer's instructions. In order to digest the purified PCR product, 45 μl of the clean-up product was then mixed with 1.5 μl (10 U/μl) of XhoI enzyme (Promega, UK), 1.5 μl (10 U/μl) of HindIII enzyme (Promega, UK), 6 μl of 10× bovine serum albumin (BSA) (Promega, UK) and 6 μl of 10× buffer B (Promega, UK) and incubated at 37° C. for 3 hours. Digestion was confirmed by agarose gel electrophoresis.

iii) Preparation of Vector.

The pRSETA system (Invitrogen, UK) was used for cloning. The plasmid was purified from overnight cultures of E. coli DH5α-pPRSETA using the Wizard® Plus SV Minipreps DNA purification system (Promega, UK) following the manufacturer's instructions. The pRSETA plasmid was digested by incubation in a 37° C. water bath for 3 hours using a reaction mixture containing 1.5 μg of pRSETA, 5 μl of 10× buffer B, 5 μl of 10× bovine serum albumin (BSA), 1 μl (10 U) of both XhoI and HindIII enzymes along with UHQ water to make up a final volume of 50 μl. To prevent recircularization and religation of linearized cloning vehicle DNA, calf intestinal alkaline phosphatase (8 μl, 1 U/μl), 10×CIAP buffer (8 μl), 10×BSA (8 μl) and UHQ water (6 μl) were added to the digestion mixture, followed by incubation at 37° C. for 30 minutes and then 56° C. for 15 minutes and purification using the Wizard® SV gel and PCR clean-up system.

iv) Ligation of Insert and Vector and Transformation into E. coli DH5α Competent Cells.

The molar ratio of insert:vector used was 3:1 and a 10 μl ligation reaction contained 10 ng of vector, appropriate amount of insert, 1 μl (=3 U) of T4 DNA ligase (Promega, UK) and 5 μl of 2× rapid ligase buffer (Promega, UK). The mixture was incubated at room temperature for 15 minutes and used for transformation, with selection on LB-ampicillin agar plates. Colonies were selected and DNA templates prepared for PCR screening for the presence of recombinant plasmids. The PCR reaction contained 12.50 GoTaq® Green Master Mix, 2.5 μl of forward and reverse primers (10 μM), 6.5 μl UHQ and 1 μl template DNA using the following conditions: 30 cycles of denaturation at 95° C., 30 s, annealing at 52° C., 30 s and extension at 72° C., 1 min. Plasmids from positive transformants were then purified using Miniprep columns for sequencing, which was done by Geneservice (Oxford, UK) with 2 primers, a T7 forward primer (T7F) and T7 reverse primer (T7R) and the resulting data were analysed by Seqman (Lasergene, DNASTAR).

v) Expression of Recombinant Protein, rNm-MIP.

The recombinant plasmid identified from sequencing reactions with the correct sequence was then transformed into competent E. coli BL21(DE3)pLysS. Transformants were selected on LB agar plates containing ampicillin (50 μg/ml) and chloramphenicol (30 μg/ml). A colony of transformed bacteria was then grown overnight in 50 ml of SOB-ampicillin (50 μg/ml)-chloramphenicol (30 μg/ml) liquid medium at 37° C. with shaking (200 rpm). Then, 40 ml of this overnight culture was inoculated in 2 L of SOB medium and the bacteria were grown until OD600 reached 0.4-0.6. Protein expression was induced by the addition of 1 mM Isopropyl-β-D-thiogalactopyranoside (IPTG) (Fisher, UK). The culture was incubated at 37° C. with shaking (200 rpm) for a further 5 hours for optimal Nm-MIP expression, as previously determined by pilot experiments, and centrifuged at 4,000 g at 4° C. for 20 minutes. As a negative control, the same strain was grown without IPTG induction. Next, the bacterial cell pellet was resuspended in lysis buffer containing 50 mM NaH₂PO₄, 300 mM NaCl and 10 mM imidazole (pH8.0) (4-5 ml/gm pellet). The bacterial suspension was frozen and thawed 3 times and sonicated (Soniprep 150 MSE, UK) on ice, using 10 s burst for 10-20 times. The lysate was then centrifuged at 10,000 g for 30 min at 4° C. In order to determine if the expressed protein was present as an insoluble protein in the cellular debris or soluble in the supernatant following the repeated freeze and thaw cycles, the pellets and supernatants were analysed by SDS-PAGE.

vi) Purification of rNm-MIP Protein.

The soluble rNm-MIP protein, found in the supernatant was purified using nickel-nitrilotriacetic acid (Ni-NTA) metal-affinity chromatography (Qiagen, Crawly, UK). Volumes of 4-5 ml of the supernatant were mixed with 1 ml of 50% (w/v) Ni-NTA resin for 30 minutes and the mixture then loaded onto a column and the flow-through collected. The column was washed with 10 column volumes of wash buffer (50 mM NaH₂PO₄, 300 mM NaCl and 20 mM imidazole, pH8.0) followed by 4 column volumes of elution buffer (50 mM NaH₂PO₄, 300 mM NaCl and 250 mM imidazole, pH8.0). Fractions of the eluate were collected and analyzed by SDS-PAGE, then pooled and dialyzed against repeated changes of phosphate buffered saline (PBS, pH7.2) containing thimerosal (1/10,000 wt/vol). Protein concentration of the dialyzed and soluble rNm-MIP was determined by BCA protein assay (Pierce, UK) and the protein was stored at −20° C. in aliquots until used.

Sequencing of PCR Product Amplified from Other Meningococcal Strains

Genomic DNA was extracted from a selection of meningococcal strains (FIG. 5) using QIAamp extraction kit according to the manufacturer's instruction (QIAamp DNA Mini and Blood Mini Handbook, Qiagen, UK). Next, the target genes were amplified by PCR with forward primer (5′-GAAACATTCAAACTCGGCTA-3′) and reverse primer (5′-GTTTCAGACGGCATTTGCCG-3′). The PCR mixture and PCR reaction was the same as for cloning Nm-MIP gene from MC58 and sequencing was done by Geneservice (Oxford, UK).

Preparation of Adjuvant and Delivery Vehicles for rNm-MIP

i) Incorporation into liposomes. Liposomes were prepared by dialysis-sonication as previously described (Humphries et al 2006. Vaccine 24: 35-44). Briefly, liposomes were prepared with L-α-phosphatidylcholine and cholesterol (Sigma-Aldrich), which were combined at a 7:2 molar ratio and dissolved (20 mg total) in 3 ml of chloroform in a glass round-bottom flask. The solvent was removed under vacuum at 25° C. with rotation to achieve an even lipid film. A solution of purified rNm-MIP (1 mg) and 100 mg of octyl β-D-glucopyranoside (Sigma-Aldrich) was prepared in 5 ml of 10 mM HEPES (Sigma-Aldrich) buffer (pH7.2) and this was then incubated at room temperature for 3 hours. The lipid film was then dissolved in this protein solution with incubation at room temperature for 1 h. Subsequently, the detergent-protein solution was extensively dialyzed against PBS containing thimerosal (1/10,000 wt/vol) for 72 h at 4° C. and then subjected to sonication (MSE Soniprep 150 sonicator; 15 μl for 20-30 bursts of 60 s each) to induce vesicle formation. Insoluble material was removed by centrifugation at 1,000 g for 10 min. Liposomes were also prepared incorporating the adjuvant monophosphoryl lipid A (MPLA; Sigma-Aldrich) at an adjuvant/protein ratio of 1:1. Control liposomes with or without MPLA were also prepared. All liposome preparations were stored in aliquots at −20° C. until used.

ii) Incorporation of rNm-MIP into Micelles.

rNm-MIP protein-Zwittergent mixtures were prepared that contained 0.5 mg of rNm-MIP ml⁻¹ PBS, 8 mg of Zwittergent 3-14 (Calbiochem, Beeston, Nottingham, United Kingdom) ml⁻¹, with or without MPLA (1 mg ml-1), and then incubated overnight at room temperature. For immunization, the solutions were diluted with saline (0.9% (w/v) NaCl) to a final concentration of 200 μg of rNm-MIP ml⁻¹ and each mouse was injected with 20 μg of protein.

For the rNm-MIP-Zw3-14 preparation containing MPLA, 0.5 mg of MPLA dissolved in PBS was used. Control micelle preparations without rNm-MIP and/or MPLA were also prepared. All micelle preparations were stored at −20° C. until used.

iii) Adsorption to Aluminium Hydroxide (Al(OH)₃; Alum).

Aluminum hydroxide gel adjuvant (2.0%) (Superfos Biosector, Denmark) was used to adsorb rNm-MIP and MC580M preparation. The mixture was composed of 500 μl of alum, 200 μg of rNm-MIP or OM in saline to make a final volume of 1 ml. Antigen was adsorbed by leaving the mixture overnight at 4° C. on an angled rotary mixer. These preparations were made fresh one day before immunization and the injection dose for each animal was 100 μl containing 20 μg protein.

iv) Saline Preparations.

As a non-adjuvant control, a solution of rNm-MIP (200 μml⁻¹) was prepared in saline and used immediately.

### Immunization of Animals

BALB/C mice (H-2^{d} haplotype) and New Zealand white rabbits were housed under standard conditions of temperature and humidity with a 12 h lighting cycle and with food and water available ad libitum. Groups of 5 mice of approximate equal size and weight were immunized intra-peritoneally with the following rNm-MIP preparations: rNm-MIP-saline, rNm-MIP-Alum, rNm-MIP-liposomes, rNm-MIP+MPLA-liposomes, rNm-MIP-Zw3-14 micelles and rNm-MIP+MPLA-zw3-14 micelles. The dose of rNm-MIP was 20 μg/mouse and the immunization schedule was 3 doses on day 0, 14 and 28. Groups of 5 mice were also injected with control preparations consisting of saline only, alum only and empty liposomes and empty Zw 3-14 micelles with and without MPLA. In addition, one group of mice was injected with OM (20 μg/mouse) adsorbed to alum and another group maintained for normal serum. Mice were terminally bled by cardiac puncture under anaesthesia on day 42.

In addition, a pair of rabbits was immunized subcutaneously with rNm-MIP emulsified in Freund's Complete Adjuvant for the primary injection and Freund's Incomplete Adjuvant for a subsequent 3 injections. Each dose contained 20 μg of protein administered at 14 day intervals. The rabbits were terminally bled from the middle ear vein and by cardiac puncture under anaesthesia 2 weeks after the last dose. To express serum, whole animal blood was allowed to clot at 37° C. for an hour and then left at 4° C. overnight. Samples were then centrifuged at 8,000 rpm for 6 minutes and the antisera were then removed and stored at −20° C. until analysed. This study complied with the animal experimentation guidelines of the UK Home Office.

Characterisation of Biological and Functional Properties of Antibodies to rNm-MIP

i) Enzyme-Linked Immunosorbent Assay (ELISA).

Flat-bottom polystyrene microtiter plates were coated overnight at 37° C. with either rNm-MIP protein or MC580M in 0.05 M sodium carbonate buffer (pH 9.6; 1 μg of protein ml⁻¹). The plate was blocked with diluent solution containing 50 mM Tris, 0.15 M NaCl, 0.05% (vol/vol) Tween 20, and 1% bovine serum albumin (wt/vol) at pH 7.4 for 1 h at 37° C. and then incubated for 1 h with serial dilutions of the murine sera. Antibody binding was detected by using anti-mouse immunoglobulin-horseradish peroxidase (HRP) conjugate (1 in 2,000 dilution from Zymed, Cambridge, United Kingdom) with 3,3′,5,5′-tetramethylbenzidine and H₂O₂ as an enzyme substrate. The reaction was quenched after 10 min with the addition of 1M H₂SO₄ and absorbance was measured at 450 nm. The ELISA titre, extrapolated from the linear portion of the serum titration curve, was taken as the reciprocal dilution which gave an increase in absorbance of 0.1 U after 10 min of substrate incubation.

ii) SDS-PAGE and Western Immunoblotting.

SDS-PAGE was performed by using a 10 to 25% linear gradient gel at 200 V for 18 h at 4° C. Samples containing rNm-MIP protein were loaded at 10-20 μg per well; OM and whole cell lysate preparations were loaded at 20 μg per well. Separated proteins were transferred to nitrocellulose by semi-dry blotting at 100 mA for 1 h and, after incubation with murine or rabbit sera, immunological reactivity was detected by using anti-mouse/rabbit immunoglobulin-alkaline phosphatase conjugate (Bio-Rad, Hemel Hempstead, United Kingdom) as described previously (Christodoulides, et al 1993 J. Gen. Microbiol. 139: 1729-1738).

iii) Immunofluorescence (IF).

Meningococci were grown overnight and harvested into PBS and suspensions (30 μl) were placed on microscope slides and left to air dry. The slides were fixed with ethanol for 15 min and then blocked with 1% (wt/vol) bovine serum albumin in PBS solution at room temperature for 30 min. Pooled murine anti-sera, diluted 1 in 100, were reacted with the fixed organisms overnight at 4° C. After a washing step with PBS, antibody bound to the meningococci was detected by reacting with anti-mouse immunoglobulin-fluorescein isothiocyanate conjugate (Dako; diluted 1 in 100 in PBS) for 1 h at room temperature in the dark. After a final wash with PBS, slides were mounted in anti-fading mountant (Dako) and viewed with a Leica SP2 confocal microscope. Reactivity was scored as previously described (Jolley et al 2001. Infect. Immun. 69: 3809-3916).

iv) Complement-Mediated Killing of Meningococci.

The bactericidal activities of pooled antisera were determined with 5% (vol/vol) baby rabbit serum as a source of exogenous complement, as previously described (Christodoulides, et al 1993 J. Gen. Microbiol. 139: 1729-1738). Murine antisera raised to purified outer membranes were used as a positive control.

v) Statistics.

Student's t-Test was used to compare differences between mean values for ELISA data sets as previously described (Christodoulides, et al 1993 J. Gen. Microbiol. 139: 1729-1738). The statistical significance of any complement-dependent bactericidal activity observed for each pooled antiserum was compared to the activity observed using decomplemented baby rabbit serum, using F- and t-tests. P values <0.05 were considered to be significant.

### Results

Cloning of the Nm-MIP Gene in E. coli Using the pRESTA System: Expression and Purification of rNm-MIP.

In order to obtain Nm-MIP free from other meningococcal components, the pRSETA vector system was used. This system has previously been used to express high levels of recombinant meningococcal PorA, PorB and Opc outer membrane proteins. A plasmid was constructed that contained the entire coding sequence for the mature Nm-MIP protein in frame with a sequence that encodes an N-terminal fusion peptide that contains six histidine residues, which function as the nickel-binding domain. Pilot expression trials demonstrated that optimal expression of recombinant (r)Nm-MIP induced by IPTG occurred by 5 h. In addition, to determine the solubility of rNm-MIP, the supernatant and pellet produced by freezing and thawing of the E. coli bacteria were analysed by SDS-PAGE. Recombinant Nm-MIP was present in the supernatant fraction (data not shown) demonstrating that the protein was soluble and could be purified therefore under native conditions.

In order to produce sufficient rNm-MIP for immunisation studies, expression and purification were scaled up to a volume of 2 L of culture medium. The rNm-MIP protein was readily purified by affinity chromatography on a Ni2+ column (FIG. 1). The native Nm-MIP protein contains 272 amino acids (FIG. 4, Sequence ID No: 1) with a predicted molecular weight of 28.9 kDa and the recombinant protein expressed in the pRSETA system, which adds 39 amino acids at the N-terminus, produces a protein of 33.4 kDa (FIG. 5, Sequence ID No: 2). SDS-PAGE showed a single homogeneous band of ˜33 kDa and fractions containing the highest levels of rNm-MIP protein were pooled and dialysed against PBS containing 0.01% (w/v) thimerosal to remove imidazole. From a typical 2 L culture volume, the yield of rNm-MIP was estimated to be approximately 74.2 mg per litre of culture.

Humoral Immune Response to rNm-MIP Protein

The purified rNM-MIP was used for immunisation studies in adjuvant formulations that are suitable for human immunisation, including adsorption to aluminium hydroxide gel and incorporation into liposomes with and without the non-toxic adjuvant monophosphoryl lipid A (MPLA). In addition, mice were immunised with protein in ZW-314 detergent micelles with and without MPLA and in saline alone. The murine humoral immune response was studied initially by reactivity of raised antisera in ELISA against purified rNm-MIP (FIG. 2). High titres of antibodies to rNm-MIP were raised using all the different adjuvant and delivery systems. rNm-MIP in detergent micelles and adsorbed to Al(OH)₃ induced statistically higher titres (×10³ geometric mean (GM) titre±95% confidence limits (CL) of 603×10³ (315×10³, 1151×10³) and 1107×10³ (790×10³, 1550×10³) respectively) than protein delivered in liposomes only (GM±95% CL of 37×10³ (4×10³, 303×10³) (p<0.05). The addition of MPLA did not significantly increase the induction of anti-rNm-MIP antibody in either detergent micelles (GM±95% CL of 1128×10³ (888×10³, 1435×10³) or liposomes (GM±95% CL of 41×10³ (8×10³, 214×10³) (p>0.05). Significantly, antibodies to rNm-MIP could be induced by immunisation with protein in saline alone (GM±95% CL of 287×10³ (100×10³, 822×10³). In addition, immunisation with MC580M also induced antibodies (GM±95% CL of 28×10³ (6×10³, 122×10³) directed against the protein (FIG. 2). Animals that were sham immunised did not produce antibodies that reacted with rNm-MIP in ELISA (FIG. 2).

### Humoral Immune Response to Nm-MIP Protein in OM

Antisera raised against rNm-MIP were also tested in ELISA against OM from the homologous strain MC58 (FIG. 2). Significant reactivity to Nm-MIP present in OM was observed with antisera raised with all the different adjuvant and delivery systems and there were no statistically significant differences between the mean antibody levels observed for any of the groups (p>0.05). As expected, significantly higher (p<0.05) anti-OM antibodies were induced by immunisation with OM compared with rNm-MIP formulae and low levels of background antibody were observed with antisera from animals that were sham immunised (FIG. 2).

The specificity of the immune response against rNm-MIP was also investigated by western blotting with whole-cell lysate and OM preparation of the homologous strain MC58 (FIG. 3). All antisera raised to rNm-MIP showed strong reactivity with a tight doublet band with an approximate molecular weight of 29 kDa and antisera from the corresponding sham immunised animals were non-reactive (FIG. 3).

### Antibody Recognition of Nm-MIP on Meningococcal Cells

Immuno-fluorescence was used to investigate the ability of antisera to recognise Nm-MIP on meningococcal cells (FIG. 7). Antisera raised against rNm-MIP in detergent micelles and liposomes alone showed weak to moderate antibody binding to meningococcal cells, but with the addition of MPLA, raised antibodies showed strong binding. Similarly, strong antibody binding was observed when sera raised to rNm-MIP adsorbed to Al(OH)₃ or in saline were reacted with bacteria (FIG. 7). All antisera from the corresponding sham immunised animals were non-reactive.

Bactericidal Activity of Antisera Raised to rNm-MIP for Homologous Strain MC58

The murine antisera were tested for their ability to promote complement-mediated killing of the homologous meningococcal strain MC58 (FIG. 8). No bactericidal activity was shown by antisera raised to rNm-MIP adsorbed to alum or incorporated in ZW-314 detergent micelles. rNm-MIP administered in liposomes alone showed bactericidal activity, with a titre of 1/32. However, the incorporation of MPLA had a significant effect on the ability of rNm-MIP to induce serum bactericidal antibodies; with both ZW-314 micelles and liposomes, bactericidal titres were increased to 1/1024. Interestingly, rNm-MIP injected in saline alone was able to induce bactericidal antibodies with a titre of 1/128, which was significantly higher than that observed with all other preparations except those containing MPLA (FIG. 8). No significant bactericidal activity was observed for antisera from sham immunised animals.

Conservation of Nm-MIP in Neisseria meningitidis

In order to investigate the conservation of Nm-MIP in Neisseria, the gene encoding the protein was amplified from a collection of carriage isolates and strains isolated from patients with disease (FIG. 6). Annotation of the amino acid sequences showed that Nm-MIP showed a high degree of conservation across the strains and three sequence types were identified. Nm-MIP of MC58 sequence type showed 100% for strains MC168, 172, 173, 174, 180 and L2470; strains within the group containing MENC11, MC90, 161, 162 and 179 and MC54 alone showed 97.8% and 98.9% similarity with MC58 respectively (FIG. 4). Notably, Nm-MIP conservation was evident amongst meningococcal strains of different group, type and sero-subtype (FIG. 6).

Antisera to rMIP (Nm-MIP) Shows Cross-Strain Bactericidal Activity.

The antisera raised against rMIP from MC58 (type I) were tested for their ability to promote complement-mediated killing of other strains from sequence types II and III (FIG. 9). Antisera from animals immunized with rMIP in saline, liposomes only, and liposomes and ZW 3-14 micelles with MPLA, which showed the highest levels of reactivity against the homologous strain MC58 (FIG. 8) were tested against heterologous strains of types II and III. For type II strain MC90, the levels of bactericidal activity were similar to those for strain MC58. In addition, for type III strain MC54, antisera raised to rMIP in saline or liposomes also showed similar bactericidal activity. By contrast, in formulations that contained MPLA, lower levels of bactericidal activity were observed with strain MC54 (FIG. 9). These results demonstrate that a recombinant meningococcal OM lipoprotein, rMIP, is able to induce antibodies with high levels of cross-strain bactericidal activity against meningococci.

Sequence Diversity of the MIP Protein Amongst Strains of Neisseria meningitidis and Other Neisseria Species.

The sequences of the MIP gene loci from a series of Neisseria strains were obtained from the NEIS1487 locus of the BIGS database at pubmlst.org. This contains sequences of the MIP gene from 206 Neisseria strains isolated from diverse geographical locations (North & South America, Western & Eastern Europe, Asia, Africa and Oceania) corresponding to N. meningitidis, N. gonorrhoeae, N. lactamica and 9 further Neisseria species. These data were combined with the sequence information from a further 7 distinct meningococcal strains to produce information on the MIP gene from a total of 213 strains. Gene sequences were translated to the inferred protein sequences using Lasergene (DNASTAR) and protein sequences compared and aligned using ClustalW (www.ebi.ac.uk).

The MIP genes from the 213 Neisseria strains were found to contain 55 different MIP sequence alleles (that is, 55 different DNA gene sequences), representing 42 distinct protein sequences (FIG. 11) within the 213 Neisseria strains. The 177 meningococcal strains which included 20 alleles, because of synonymous substitutions, represented only 11 distinct protein sequence types. Of these, 8 sequence types were found among the 90 serogroup B meningococci analysed (FIG. 11).

Comparison of the 11 aligned meningococcal protein sequences revealed a high degree of conservation with 95-99% sequence identity between the strains (FIG. 12). The protein sequence type corresponding to allele 2 Seq ID No: 1) was found in 106 of 177 meningococcal strains (59.9%) and was even more highly conserved in the 90 serogroup B strains (76%) (FIG. 13). This sequence type corresponds to the protein sequence designated Type I and present in the strain MC58. The region of the proteins with greatest sequence diversity corresponded to positions 28-31 of the translated allele 2 protein sequence, with three sequence types having a deletion of 5 amino acids and one sequence type containing an insertion of one residue and an adjacent substitution. The remaining 7 sequence types were identical in this region with diversity being generated by occasional amino acid substitutions elsewhere throughout the protein.

In FIG. 12 the sequence of allele 02 corresponds to Seq ID No: 1, the sequence of allele 3 corresponds to Seq ID No: 3, the sequence of allele 11 corresponds to Seq ID No: 4, the sequence of allele 13 corresponds to Seq ID No: 5, the sequence of allele 05 corresponds to Seq ID No: 6, the sequence of allele 06 corresponds to Seq ID No: 7, the sequence of allele 24 corresponds to Seq ID No: 8, the sequence of allele 15 corresponds to Seq ID No: 9, the sequence of allele 07 corresponds to Seq ID No: 10, the sequence of allele 01 corresponds to Seq ID No: 11, the sequence of allele 22 corresponds to Seq ID No: 12.

The 11 N. meningitidis protein sequence types were unique and were not shared with of the other species including N. gonorrhoeae, which itself comprised 3 distinct sequence types that were also unique.

Analysis of a total of 13 meningococcal strains identified 3 sequence types designated Types I, II and III which correspond to the translated alleles 2, 1 and 6 respectively. Antisera raised against a recombinant protein designated Type I showed bactericidal activity against the homologous strain and also against heterologous strains representing Types II and III. These three sequence types account for 76% of the 177 meningococcal strains and 89% of serogroup B meningococci analysed. Of particular note, Type II contains the deletion corresponding to positions 28-31 demonstrating that variation in the region of greatest sequence diversity does not influence the potential protective bactericidal effect.

1. An immunogenic composition comprising the Nm-MIP protein or an immunogenic part thereof or an analogue thereof, wherein the composition is capable of eliciting an immune response when administered to a human or non-human animal.

2. The composition of claim 1 which is capable of eliciting an immune response against one or more of N. meningitidis serogroup B strains, N. meningitidis serogroup A strains, N. meningitidis serogroup C strains, N. meningitidis serogroup W135 strains, N. meningitidis serogroup Y strains and N. gonorrhoeae.

3. The composition of any preceding claim wherein the immune response elicited affects the ability of N. meningitidis and/or N. gonorrhoeae to infect an immunised animal.

4. The composition of any preceding claim wherein the Nm-MIP protein is a recombinant protein.

5. The composition of any preceding claim wherein the Nm-MIP protein has a sequence with at least 50% homology with the sequence of Seq ID No: 1, 2, 3, 4, 5, 6, 7, 8, 9, 10, 11 or 12 or an immunogenic part thereof or an analogue thereof.

6. The composition of any preceding claim wherein the Nm-MIP protein has a sequence with at least 80% homology with the sequence of Seq ID No: 1, 2, 3, 4, 5, 6, 7, 8, 9, 10, 11 or 12 or an immunogenic part thereof or an analogue thereof.

7. The composition of any preceding claim wherein the Nm-MIP protein has at least 90% homology with the sequence of Sequence ID No: 1 or Sequence ID No: 2.

8. The composition of any preceding claim wherein the Nm-MIP protein has a sequence with at least 95% homology with the sequence of Seq ID No: 1, 2, 3, 4, 5, 6, 7, 8, 9, 10, 11 or 12 or an immunogenic part thereof or an analogue thereof.

9. The composition of any preceding claim wherein the Nm-MIP protein has the sequence of Sequence ID No: 1, 2, 3, 4, 5, 6, 7, 8, 9, 10, 11 or 12.

10. The composition of any preceding claim wherein further comprising one or more antigens, in addition to the Nm-MIP protein or an immunogenic part thereof or an analogue thereof.

11. The composition of any preceding claim wherein the immune response elicited is a protective immune response.

12. The composition of any preceding claim wherein the composition is incorporated into liposomes or detergent micelles for administration.

13. The composition of any preceding claim further comprising an adjuvant.

14. The composition of claim 13 wherein the adjuvant is monophosphoryl Lipid A.

15. A pharmaceutical composition comprising a composition of any preceding claim and a pharmaceutically acceptable carrier or excipient.

16. A vaccine composition comprising the composition of any preceding claim.

17. The vaccine composition of claim 16 for the prevention of N. meningitidis and/or Neisseria gonorrhoeae infection.

18. The vaccine composition of claim 17 for the prevention of N. meningitidis serogroup B and/or serogroup A and/or serogroup C and/or serogroup W135 and/or serogroup Y infection.

19. The use of the Nm-MIP protein or an immunogenic part thereof or an analogue thereof in the preparation of a medicament for eliciting an immune response.

20. The use of claim 19 wherein the medicament is for use in the prophylactic or therapeutic vaccination of subjects against N. meningitidis and/or N. gonorrhoeae.

21. The use of claim 19 or 20 wherein the Nm-MIP protein is as defined in any of claims 4 to 9.

22. A composition comprising the Nm-MIP protein or an immunogenic part thereof or an analogue thereof for use in generating an immune response to N. meningitidis and/or N. gonorrhoeae.

23. A method of protecting a human or non-human animal from the effects of infection by N. meningitidis and/or N. gonorrhoeae comprising administering to the human or non-human animal a composition according to any of claims 1 to 18.

24. A kit for use in inducing an immune response in an organism, comprising an immunogenic or vaccine composition according to any of claims 1 to 18 and instructions relating to administration.


SEQUENCE LISTING (12 sequences)

SEQ ID NO: 1 - protein, 272 aa, Neisseria meningitidis
MNTIFKISALTLSAALALSACGKKEAAPASASEPAAASSAQGDTSSIGSTMQQASYAMGVDIGRSLKQMKEQGAEIDLKVFTEAMQAVYDGKEIKMTEEQAQEVMMKFLQEQQAKAVEKHKADAKANKEKGEAFLKENAAKDGVKTTASGLQYKITKQGEGKQPTKDDIVTVEYEGRLIDGTVFDSSKANGGPVTFPLSQVIPGWTEGVQLLKEGGEATFYIPSNLAYREQGAGDKIGPNATLVFDVKLVKIGAPENAPAKQPAQVDIKKVN

SEQ ID NO: 2 - protein, 311 aa, Neisseria meningitidis
MRGSHHHHHHGMASMTGGQQMGRDLYDDDDKDRWGSELEMNTIFKISALTLSAALALSACGKKEAAPASASEPAAASSAQGDTSSIGSTMQQASYAMGVDIGRSLKQMKEQGAEIDLKVFTEAMQAVYDGKEIKMTEEQAQEVMMKFLQEQQAKAVEKHKADAKANKEKGEAFLKENAAKDGVKTTASGLQYKITKQGEGKQPTKDDIVTVEYEGRLIDGTVFDSSKANGGPVTFPLSQVIPGWTEGVQLLKEGGEATFYIPSNLAYREQGAGDKIGPNATLVFDVKLVKIGAPENAPAKQPAQVDIKKVN

SEQ ID NO: 3 - protein, 272 aa, Neisseria meningitidis
MNTIFKISALTLSAALALSACGKKEAAPASASEPAAASSAQGDTSSIGSTMQQASYAMGVDIGRSLKQMKEQGAEIDLKVFTEAMQAVYDGKEIKMTEEQAQEVMMKFLQEQRAKAVEKHKADAKANKEKGEAFLKENAAKDGVKTTASGLQYKITKQGEGKQPTKDDIVTVEYEGRLIDGTVFDSSKANGGPVTFPLSQVILGWTEGVQLLKEGGEATFYIPSNLAYREQGAGDKIGPNATLVFDVKLVKIGAPENAPAKQPAQVDIKKVN

SEQ ID NO: 4 - protein, 272 aa, Neisseria meningitidis
MNTIFKISALTLSAALALSACGKKEAAPASASEPAAASSAQGDTSSIGSTMQQASYAMGVDIGRSLKQMKEQGAEIDLKVFTEAMQAVYDGKEIKMTEEQAQEVMMKFLQEQQAKAVEKHKADAKANKEKGEAFLKENAAKDGVKTTASGLQYKITKQGEGKQPTKDDIVTVEYEGRLIDGTVFDSSKANGGPVTFPLSQVILGWTEGVQLLKEGGEATFYIPSNLAYREQGAGDKIGPNATLVFDVKLVKIGAPENAPAKQPAQVDIKKVN

SEQ ID NO: 5 - protein, 272 aa, Neisseria meningitidis
MNTIFKISALTLSAALALSACGKKEAAPASASEPAAASSAQGDTSSIGSTMQQASYAMGVDIGRSLKQMKEQGAEIDLKVFTEAMQAVYDGKEIKMTEEQAQEVMMKFLQEQQAKAVEKHKADAKANKEKGEAFLKENAAKDGVKTTASGLQYKITKQGEGKQPTKDDIVTVEYEGRLIDGTVFDSSKANGGTVTFPLSQVIPGWTEGVQLLKEGGEATFYIPSNLAYREQGAGDKIGPNATLVFDVKLVKIGAPENAPAKQPAQVDIKKVN

SEQ ID NO: 6 - protein, 272 aa, Neisseria meningitidis
MNTIFKISALTLSAALALSACGKKEAAPASASEPAAASSAQDDTSSIGSTMQQASYAMGVDIGRSLKQMKEQGAEIDLKVFTEAMQAVYDGKEIKMTEEQAQEVMMKFLQEQQAKAVEKHKADAKANKEKGEAFLKENAAKDGVKTTASGLQYKITKQGEGKQPTKDDIVTVEYEGRLIDGTVFDSSKANGGPVTFPLSQVIPGWTEGVQLLKEGGEATFYIPSNLAYREQGAGDKIGPNATLVFDVKLVKIGAPENAPAKQPAQVDIKKVN

SEQ ID NO: 7 - protein, 272 aa, Neisseria meningitidis
MNTIFKISALTLSAALALSACGKKEAAPASASEPAAASSAQGDTSSIGSTMQQASYAMGVDIGRSLKQMKEQGAEIDLKVFTEAMQAVYDGKEIKMTEEQAQEVMMKFLQEQQAKAVEKHKADAKANKEKGEAFLKENAGKESVKTTASGLQYKITKQGEGKQPTKDDIVTVEYEGRLIDGTVFDSSKANGGPVTFPLSQVIPGWTEGVQLLKEGGEATFYIPSNLAYREQGAGDKIGPNATLVFDVKLVKIGAPENAPAKQPAQVDIKKVN

SEQ ID NO: 8 - protein, 272 aa, Neisseria meningitidis
MNKIFKISTLTLAATLALSACGKKEAAPASASEPAAASAAQGDTSSIGSTMQQASYAMGVDIGRSLKQMKEQGAEIDLKVFTEAMQAVYDGKEIKMTEEQAQEVMMKFLQEQQAKAVEKHKADAKANKEKGEAFLKENAAKDGVKTTASGLQYKITKQGEGKQPTKDDIVTVEYEGRLIDGTVFDSSKANGGPVTFPLSQVIPGWTEGVQLLKEGGEATFYIPSNLAYREQGAGDKIGPNATLVFDVKLVKIGAPENAPAKQPAQVDIKKVN

SEQ ID NO: 9 - protein, 273 aa, Neisseria meningitidis
MNTIFKISALTLSAALALSACGKKEAAPAATASEPAAASAAQGDTSSIGSTMQQASYAMGVDIGRSLKQMKEQGAEIDLKVFTEAMQAVYEGKEIKMTEEQAQEVMMKFLQEQQAKAVEKHKADAKANKEKGEAFLKENAAKEGVKTTASGLQYKITKQGEGKQPTKDDIVTVEYEGRLIDGTVFDSSKANGGPATFPLSQVIPGWTEGVQLLKEGGEATFYIPSNLAYREQGAGEKIGPNATLVFDVKLVKVGAPENASAQQPVQVDVKKVN

SEQ ID NO: 10 - protein, 268 aa, Neisseria meningitidis
MNTIFKISALTLSAALALSACGKKEAASEPAAASAAQGDTSSIGSTMQQASYAMGVDIGRSLKQMKEQGAEIDLKVFTEAMQAVYDGKEIKMTEEQAQEVMMKFLQEQQAKAVEKHKAEAKANKEKGEAFLKENAAKDGVKTTASGLQYKITKQGEGKQPTKDDIVTVEYEGRLIDGTVFDSSKANGGPVTFPLSQVIPGWTEGVQLLKEGGEATFYIPSNLAYREQGAGDKIGPNATLVFDVKLVKIGAPENAPAKQPAQVDIKKVN

SEQ ID NO: 11 - protein, 268 aa, Neisseria meningitidis
MNTIFKISALTLSAALALSACGKKEAASEPAAASAAQGDTSSIGSTMQQASYAMGVDIGRSLKQMKEQGAEIDLKVFTEAMQAVYDGKEIKMTEEQAQEVMMKFLQEQQAKAVEKHKADAKANKEKGEAFLKENAAKDGVKTTASGLQYKITKQGEGKQPSKDDIVTVEYEGRLIDGTVFDSSKANGGPVTFPLSQVIPGWTEGVQLLKEGGEATFYIPSNLAYREQGAGDKIGPNATLVFDVKLVKIGAPENAPAKQPAQVDIKKVN

SEQ ID NO: 12 - protein, 268 aa, Neisseria meningitidis
MNTIFKISALTLSAALALSACGKKEAASEPAAASAAQGDTSSIGSTMQQASYAMGVDIGRSLKQMKEQGAEIDLKVFTEAMQAVYDGKEIKMTEEQAQEVMMKFLQEQQAKAVEKHKADAKANKEKGEAFLKENAAKDGVKTTASGLQYKITKQGEGKQPTKDDIVTVEYEGRLIDGTVFDSSKANGGPVTFPLSQVIPGWTEGVQLLKEGGEATFYIPSNLAYREQGAGDKIGPNATLVFDVKLVKIGAPENAPAKQPAQVDIKKVN